(19) United States
(12) Patent Application Publication
(10) Pub. No.: US 2026/0232990 A1
(43) Pub. Date: Aug. 13, 2026

(54) BLOOD PUMP SYSTEM

(71) Applicant: Abiomed Europe GmbH, Aachen (DE)

(72) Inventors: Katrin Lunze, Aachen (DE); Christoph Nix, Aachen (DE)

(73) Assignee: Abiomed Europe GmbH, Aachen (DE)

(21) Appl. No.: 19/153,889

(22) PCT Filed: Feb. 5, 2024
(86) PCT No.: PCT/EP2024/052767

§ 371 Date: Aug. 5, 2025

(30) Foreign Application Priority Data
Feb. 7, 2023 (EP) 23155389.2

(51) Int. Cl. A61M 60/515; A61M 60/122; A61M 60/216

(52) U.S. Cl. CPC A61M 60/515; A61M 60/122; A61M 60/216

(57) ABSTRACT

A blood pump system that has a blood pump having a pump housing with a blood flow inlet and a blood flow outlet connected by a passage, a pump element disposed in said pump housing, a drive unit configured to drive the pump element at a set speed (Nₛₑₜ) so that a flow is generated between the blood flow inlet and the blood flow outlet; a sensor arrangement configured to sense at least one circulatory system related parameter (CSRP) at least intermittently; a controller configured to process the at least one circulatory system related parameter (CSRP); and a storage device configured to continuously store the processed at least one circulatory system related parameter; wherein the a controller is further configured to execute a weaning procedure in that i) a first sequence of the processed at least one circulatory system related parameter (CSRP) is stored in the storage device for a first time interval; ii) a first average value (AV₁) for the first sequence is calculated; iii) the set speed (Nₛₑₜ) is reduced by a speed reduction value (dN_{R}) after the first average value (AV₁) is calculated; iv) a second sequence of the processed at least one circulatory system related parameter (CSRP) is stored in the storage device for a second time interval; v) a second average value (AV₂) for the second sequence is calculated; vi) an comparison value (|Diff|) of the difference of the first average value (AV₁) and the second average value (AV₂) is compared with a predefined threshold value (TR_{CSRP}); vii) wherein the controller is configured to repeat steps iv) to vi), and configured to increase a first counter (Cᵤₚ) by 1 provided the comparison value (|Diff|) is within the predefined threshold value (TR_{CSRP}), and configured to increase a second counter (C_{down}) by 1 provided the comparison value (|Diff|) is outside the predefined threshold value (TR_{CSRP}); and wherein the controller is configured to repeat steps i) to vii) and configured to set the first counter (Cᵤₚ) to zero when the first counter (Cᵤₚ) reaches a predefined first counter maximum value (TR_{Cup}), and wherein the controller is configured to increase the set speed (Nₛₑₜ) by a speed increase value (dN₁) and configured to set the second counter (C_{down}) to zero and configured to repeat steps iv) to vii) when the second counter (C_{down}) reaches a predefined second counter maximum value (TR_{Cdown}).

The present invention relates to a blood pump system. In particular, the present invention relates to a blood pump system comprising a blood pump and in particular an intravascular blood pump for percutaneous insertion into a patient's blood vessel, to support a blood flow in a patient's blood vessel. The blood pump may also be an intracardiac blood pump, intravascular blood pump or any other kind of ventricular support device.

### BACKGROUND OF THE INVENTION

Various blood pumps are known from the prior art e.g., axial blood pumps, centrifugal (i.e. radial) blood pumps or mixed-type blood pumps, where the blood flow is caused by axial forces as well as by radial forces. Such blood pumps may be introduced into the heart of a patient to support the blood flow from the heart into an artery e.g., the aorta. The blood pump may be introduced percutaneously during a cardiac procedure through the vascular system, such as by a catheterization procedure. After the blood pump has been placed, blood is unloaded by the blood pump from the left ventricle into the aorta to restore adequate systemic blood flow. Therefore, a blood pump typically comprises a pump housing having a blood flow inlet and a blood flow outlet connected by a passage, a pump element in form of an impeller disposed in said pump housing and a drive unit configured to drive the impeller at an actual speed so that a flow of blood is generated between the blood flow inlet and the blood flow outlet. Usually, a cannula is attached to the blood flow inlet. The cannula reaches through e.g., the aortic valve into the left ventricle and blood is unloaded from the left ventricle through the cannula and exits the pump housing through the blood flow outlet into the aorta. Of course, such blood pumps can also be used for cardiac assist of the right ventricle of the heart, where the blood pump unloads blood from the right ventricle or right atrium into the pulmonary artery or unloads blood from the venous system into the patient's heart.

Hence, such blood pumps are intended to support the function of a patient's heart, either in a short-term application, in which an intravascular blood pump is placed in the patient for a couple of days or weeks, or in a long-term application, in which the intravascular blood pump is placed in the patient for a couple of weeks or months.

However, an intravascular blood pump may not be switched off immediately at an expected end of the therapy. Rather, a controlled weaning of the patient from the intravascular blood pump needs to be performed. There are no established methods to do so, however, and the applied weaning is carried out manually and largely depends on the skills and experiences available in the medical facility or center taking care of the patient. Failure during the weaning can mean a setback in the success of the therapy and may distinctly prolong the necessary time for the therapy until a next weaning can be performed. Of course, for the sake of the patient, this should be avoided.

Hence, it is an objective of the present disclosure to provide a blood pump system which facilitates the weaning of the patient from a blood pump and which greatly reduces the risk of a setback in the success of the therapy.

### SUMMARY OF THE INVENTION

According to a first aspect, the blood pump system comprises a blood pump, a sensor arrangement, a controller and a storage device. The blood pump has a pump housing with a blood flow inlet and a blood flow outlet connected by a passage. A pump element is disposed in said pump housing. The blood pump further comprises a drive unit configured to drive the pump element at a set speed so that a flow is generated between the blood flow inlet and the blood flow outlet. The sensor arrangement is configured to sense at least one circulatory system related parameter at least intermittently. The sensor arrangement may be configured to sense the at least one circulatory system related parameter continuously. The sensor arrangement may be an intracorporeal sensor arrangement, an extracorporeal sensor arrangement or a combination thereof. For instance, the blood pump may comprise the sensor arrangement in which sensors are positioned, e.g., at the pump housing. The sensors may be optical sensors.

The blood pump system further comprises a controller configured to process the at least one circulatory system related parameter and a storage device configured to continuously store the processed at least one circulatory system related parameter. The storage device may also be configured to continuously store the sensed at least one circulatory system related parameter directly.

The blood pump according to the present invention may correspond to the aforementioned blood pump. Hence, the blood pump may be an intravascular blood pump or an intracardiac blood pump.

The controller is further configured to execute a weaning procedure in that
i) a first sequence of the processed at least one circulatory system related parameter is stored in the storage device for a first time interval;
ii) a first average value for the first sequence is calculated;
iii) the set speed is reduced by a speed reduction value after the first average value is calculated;
iv) a second sequence of the processed at least one circulatory system related parameter is stored in the storage device for a second time interval;
v) a second average value for the second sequence is calculated;
vi) a comparison value of the difference of the first average value and the second average value is compared with a predefined threshold value;
vii) wherein the controller is configured to repeat steps iv) to vi), and
   configured to increase a first counter by 1 provided the comparison value is within the predefined threshold value, and
   configured to increase a second counter by 1 provided the comparison value is outside the predefined threshold value.

The controller is further configured to repeat steps i) to vii) and configured to set the first counter to zero when the first counter reaches a predefined first counter maximum value. The controller is further configured to increase the set speed by a speed increase value and configured to set the second counter to zero and configured to repeat steps iv) to vii) when the second counter reaches a predefined second counter maximum value.

The weaning procedure executed by the controller thus provides an automated weaning of the patient from the blood pump and reduces the risk of a setback in the success of the therapy, as the speed of the drive unit is rapidly increased again if necessary, namely in case the second counter reaches the predefined second counter maximum. Further, when the sensed and then processed circulatory system related parameter after the reduction in speed is within the predefined threshold, it is assumed that the speed reduction does not lead to an instable patient. Rather, it is then assumed that the patient is still stable despite the reduced flow of the blood pump caused by the speed reduction of the drive unit.

A circulatory system related parameter in sense of the present disclosure is any parameter or value measurable and related to the organs of the patient, like heart, blood vessels and blood. Preferably, the at least one sensed or processed circulatory system related parameter is a cardiovascular system related parameter.

It has to be noted that the threshold value according to the disclosure may comprise a single threshold, multiple different thresholds and/or a threshold range. Furthermore, it is preferable that the first counter and the second counter are both set to zero when the weaning procedure is initiated for the first time. The threshold value may be predefined by the physician overseeing the weaning procedure. Preferably, the comparison value is an absolute value.

Preferably, the controller is configured to end the weaning procedure when the set speed reaches a predefined minimum speed or a predefined maximum speed. The predefined minimum speed may be a speed where the flow through the blood pump is very low e.g., 0.1 l/min or less. When the set speed reaches the minimum speed, it is assumed that the patient is weaned and the blood pump may be explanted from the patient's body. The predefined maximum speed may correspond to the set speed before the weaning procedure is initiated for the first time. In case the set speed reaches the maximum speed, it is assumed that the patient is not ready to wean and still needs full support of the blood pump. The minimum speed and the maximum speed may be predefined by the physician overseeing the weaning procedure. Instead of defining a minimum speed and a maximum speed, a minimum blood flow of the blood pump and a maximum blood flow of the blood pump may be defined. In this case, the set speed corresponds to a set blood flow of the blood pump. The controller may be configured to convert a speed to a blood flow and vice versa. In doing so, the controller may be configured to take into account influencing parameters based e.g., on motor currents or data received from the sensor arrangement.

Preferably, the processed at least one circulatory system related parameter comprises one or more of a heart rate, a heart rate variance (sometimes also called heart rate variability), a mean arterial pressure, a left ventricular end diastolic pressure and a blood flow of the blood pump. The parameters may either be directly sensed by the sensor arrangement or may be derived from the sensed parameters by the controller. In case the parameters are directly used, the at least one sensed circulatory system related parameter corresponds to the at least one processed circulatory system related parameter. Further, the controller may be configured to consider fluctuations in the at least one circulatory system related parameter caused e.g., by breathing or ventilation of the patient. In this regard, the predefined threshold value may comprise one or more of a threshold value for the heart rate, a threshold value for the heart rate variance, a threshold value for the mean arterial pressure, a threshold value for the left ventricular end diastolic pressure and a threshold value for the blood flow of the blood pump.

Preferably, the first counter maximum value is larger than the second counter maximum value. Thus, the time interval for a speed increase is shorter than the time interval for the speed decrease during the weaning procedure. Accordingly, in case the patient becomes instable after a speed decrease a respective speed increase is triggered within a comparably short period of time. Further, a possible further speed decrease is only triggered in case the patient is stable over a comparably longer period of time. In other words, a speed increase is much faster than a speed decrease.

The first time interval may be larger than the second time interval. Accordingly, the first average value is based on a larger population of the processed at least one circulatory system related parameter compared to the second average value. Thus, the weaning procedure is based on a stable comparative value to decide whether the set speed must be increased or decreased.

Preferably, the predefined threshold value for the processed at least one circulatory system related parameter comprises a lower threshold range and an upper threshold range. The controller may thus be configured to set the speed reduction value to a first speed reduction value when the comparison value is within the lower threshold range, and configured to set the speed reduction value to a second speed reduction value when the comparison value is within the upper threshold range. Preferably, the first speed reduction value is larger than the second speed reduction value. Accordingly, the to be applied speed reduction value may be set depending on the variation between the comparison value and the threshold value. Accordingly, in case of a comparatively small variation, the set speed is reduced by a higher value, namely the first speed reduction value, than it is the case for an increased variation, when the set speed is reduced by a lower value, namely the second speed reduction value.

Preferably, the lower threshold range is ±5 mmHg for circulatory system related parameters depicting a mean arterial blood pressure, ±2.5 mmHg for circulatory system related parameters depicting a left ventricular end diastolic pressure, ±10 bpm for circulatory system related parameters depicting a heart rate and ±150 msec for circulatory system related parameters depicting a heart rate variance. Accordingly, the upper threshold range may be ±10 mmHg, ±5 mmHg, ±20 bpm and ±75 msec respectively.

For instance, the speed reduction value may be in a range between 100 rpm and 600 rpm, wherein the first speed reduction value may be in a range between 400 rpm to 600 rpm and the second speed reduction value may be in a range between 100 rpm and 300 rpm. Accordingly, the first speed reduction value may be set to 500 rpm and the second speed reduction value may be set to 200 rpm. The speed reduction value and the first speed reduction value and second speed reduction value may be predefined by the physician overseeing the weaning of the patient from the blood pump.

The controller may further be configured to set the speed increase value to a first speed increase value or to a second speed increase value based on e.g., the distance between the comparison value and the threshold value and/or based on the value of the second counter and/or based on the second average value. Preferably, the first speed increase value is smaller than the second speed increase value. Consequently, the extent of the patient's instability is taken into account to set the new and increased set speed. In other words, in case a more severe instability of the patient is detected during the weaning procedure the set speed is increased by a higher value, namely the second speed increase value.

For instance, the speed increase value may be in a range between 400 rpm and 1,500 rpm, wherein the first speed increase value may be in a range between 400 rpm to 600 rpm and the second speed increase value may be in a range between 800 rpm and 1,200 rpm. Accordingly, the first speed increase value may be set to 500 rpm and the second speed increase value may be set to 1,000 rpm. The speed increase value and the first speed increase value and second speed increase value may be predefined by the physician overseeing the weaning of the patient from the blood pump.

The first time interval and the second time interval may be based on a number of heart beats. Alternatively, the first time interval and the second time interval may be based on a time span or on a number of values for the first sequence and the second sequence respectively. For instance, the first sequence may comprise a processed circulatory system related parameter for a timepoint every two seconds over a total time span of 30 minutes, i.e. a total of 900 consecutive values. For instance, the second sequence may comprise a processed circulatory system related parameter for a timepoint every two seconds over a total time span of ten seconds, i.e. a total of five consecutive values.

Preferably, the controller is further configured to execute an outlier identification in the first sequence of the processed at least one circulatory system related parameter and/or in the second sequence of the processed at least one circulatory system related parameter. The outlier identification may be based on common statistical methods, like Grubb's test or Student's t-test.

The controller may further be configured to alter the set speed to avoid regurgitation through the blood pump. This is of particular relevance when the set speed is close to or at the predefined minimum speed for intravascular or intracardiac blood pumps. As those blood pumps reach through a cardiac valve (e.g., the aortic valve) an open path through said valve is established which allows for backflow when the blood pump is not driven or driven at a very low speed, as the pressure within the blood vessel is higher than the pressure in the ventricle during the diastolic phase. Accordingly, the controller may be configured to account therefore and to avoid the regurgitation. A particularly suitable control method is disclosed in WO 2019/034775 A1 of the applicant which is hereby incorporated by reference in its entirety.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the drawings:

FIG. 1a shows a schematic overview of a blood pump system according to a first embodiment,

FIG. 1b shows a schematic overview of a blood pump system according to a second embodiment, and

FIG. 2 is a flow chart of an exemplary weaning procedure.

### DETAILED DESCRIPTION

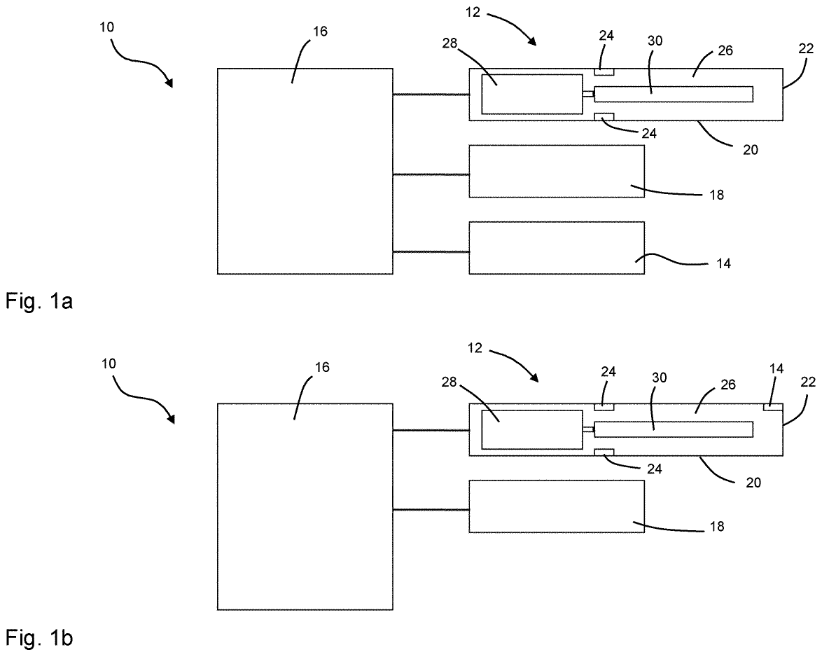

FIG. 1a shows a schematic overview of a blood pump system 10 according to a first embodiment. The blood pump system 10 comprises a blood pump 12, a sensor arrangement 14, a controller 16 and a storage device 18. The blood pump 12, the sensor arrangement 14 and the storage device 18 are connected to the controller 16 by suitable devices e.g., by cables. Of course, wireless connections are also possible. In addition, the storage device 18 may be an integral part of the controller 16.

The blood pump 12 may be an intravascular blood pump belonging to the Impella® product family of the applicant. The blood pump 12 comprises a pump housing 20 with a blood flow inlet 22 and a blood flow outlet 24. The blood flow inlet 22 is connected to the blood flow outlet 24 by a passage 26. Here, the pump housing 20 comprises a plurality of blood flow outlets 24 evenly distributed around the circumference of the blood pump housing 20.

A drive unit 28 is disposed within the pump housing 20. The drive unit 28 is configured to drive a pump element 30 in form of an impeller disposed in the pump housing 20 at a set speed Nₛₑₜ, as will be explained in more detail below. Although the drive unit 28 is disposed in the pump housing 20 in the embodiment shown, it is also possible that the drive unit 28 is extracorporeally provided and connected to the impeller 30 by a suitable device.

Rotation of the impeller 30 may be caused by a direct connection between the drive unit 28 and the impeller 30 or an indirect connection, like a magnetic coupling. When the impeller 30 rotates, a blood flow is generated between the blood flow inlet 22 along the passage 26 to the blood flow outlet 24. In particular, when the blood pump 12 is used as left ventricular support, the blood flow inlet 22 is positioned so that blood from the left ventricle of the patient's heart can be drawn into the blood pump 12 and can exit the blood pump 12 through the blood flow outlet 24 into the aorta. Of course, the blood pump 12 may also be used as right ventricular support to deliver blood from the venous system into the patient's heart or to unload blood from the patient's heart into the pulmonary artery.

FIG. 1b shows a schematic overview over a blood pump system 10 according to a second embodiment. The blood pump system 10 according to the second embodiment differs from the blood pump system according to the first embodiment in that the blood pump 12 comprises the sensor arrangement 14. Of course, it is also possible that the blood pump 12 comprises only a part of the sensor arrangement. For instance, a first sensor may be supported by the pump housing 20 so as to measure an artery pressure. A second sensor may also be supported by the pump housing 20 so as to measure a left ventricular pressure. Alternatively, the first and/or the second sensor may be remotely placed from the blood pump 12 so as to measure parameters of interest. I heart rate may be computed by the controller 16 from a suitable signal, like an ECG signal, motor current or blood pressure. It has to be mentioned in this regard that the heart rate may also be called pulse rate in case it is determined based on a pressure signal. However, in the following only the term heart rate will be used.

In the embodiments shown in FIGS. 1a and 1b, the sensor arrangement 14 is configured to sense at least one circulatory system related parameter CSRP. In particular, the sensor arrangement 14 may be configured to continuously or intermittently sense the at least one circulatory system related parameter CSRP. The at least one circulatory system related parameter CSRP preferably is at least one cardiovascular system related parameter and may comprise one or more of a heart rate HR, a heart rate variance HRV, a mean arterial pressure MAP and a left ventricular end diastolic pressure LVEDP.

The sensed and output at least one circulatory system related parameter CSRP by the sensor arrangement 14 either directly delivers the at least one circulatory system related parameter CSRP of interest or is further processed by the controller 16 to deliver the processed at least one circulatory system related parameter CSRP of interest. In any case, the data output by the sensor arrangement 14 and the data processed by the controller 16 can be stored in the storage device 18. In particular, the storage device 18 is configured to store several sequences of the data received from the controller 16. Preferably, the storage device 18 continuously stores the data and the controller 16 is configured to retrieve the required data from the storage device 18 as will be described in more detail below.

In the following, the disclosure will be described for a blood pump 12 to be used as a left ventricular support. Of course, the present disclosure is also usable with a blood pump to be used as a right ventricular support.

As mentioned above, the blood pump 12 is intended to support the function of a patient's heart, either in a short-term application, in which an intravascular blood pump is placed in the patient for a couple of days or weeks, or in a long-term application, in which the blood pump 12 is placed in the patient for a couple of weeks or months. At an expected end of the therapy, the blood pump 12 may not be switched off and explanted immediately. Rather, the patient is to be weaned from the blood pump 12.

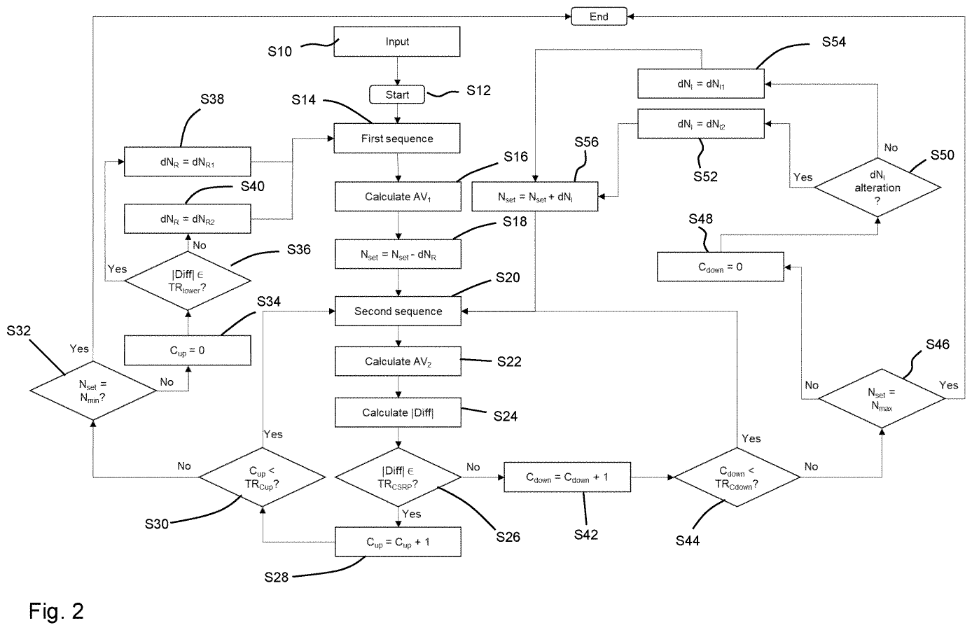

Therefore, the controller 16 is configured to execute a weaning procedure as will be described in the following with reference to the flow chart shown FIG. 2.

Prior to starting the weaning procedure, the physician or medical staff inputs parameters intended for the weaning of the patient in step S10. In particular, the physician inputs age and gender of the patient and further defines a minimum speed Nₘᵢₙ, a maximum speed Nₘₐₓ, a first speed reduction value dN_{R1}, a second speed reduction value dN_{R2}, a first speed increase value dN_{I1}, a second speed increase value dN_{I2} and different threshold values TR_{CSRP}. To do so, the physician uses a suitable input device of the blood pump system 10 e.g., a tablet computer, a touchscreen, a keyboard or the like.

The minimum speed Nₘᵢₙ depicts that speed of the pump element 30 at which the patient is assumed to be weaned off the device. Preferably, the blood pump 12 generates a minimum blood flow of 0.1 l/min or less when operated at the minimum speed Nₘᵢₙ. The maximum speed Nₘₐₓ depicts that speed of the pump element 30 at which the patient is assumed to be not ready to be weaned off and still needs support of the blood pump 12. Preferably, the maximum speed Nₘₐₓ corresponds to the current speed applied prior to the start of the weaning process i.e., a speed at which the condition of the patient is stable. This speed may for instance be 25,000 rpm. The first speed reduction value dN_{R1} preferably is in a range between 400 rpm to 600 rpm. In the exemplary embodiment described in the following, the first speed reduction value dN_{R1} is set at 500 rpm. The second speed reduction value dNᵣ₂ preferably is in a range between 100 rpm to 300 rpm. In the exemplary embodiment described in the following, the second speed reduction value dN_{R2} is set at 200 rpm. The first speed increase value dN_{I1} preferably is in a range between 400 rpm to 600 rpm. In the exemplary embodiment described in the following, the first speed increase value dN_{I1} is set at 500 rpm. The second speed increase value dN_{I2} preferably is in a range between 800 rpm and 1,200 rpm. In the exemplary embodiment described in the following, the second speed increase value dN_{I2} is set at 1,000 rpm. Depending on which circulatory system related parameters CSRP are monitored for applying the weaning procedure, the physician sets threshold values TR_{CSRP} for every parameter. In the following exemplary embodiment, four circulatory system related parameters CSRP are used, namely the heart rate HR, the heart rate variance HRV, the mean arterial pressure MAP and the left ventricular end diastolic pressure LVEDP. Thus, respective threshold values are defined, namely a threshold value for the heart rate TR_{HR}, a threshold value for the heart rate variance TR_{HRV}, a threshold value for the mean arterial pressure TR_{MAP} and a threshold value for the left ventricular end diastolic pressure TR_{LVEDP}. Preferably, the threshold values are ±10 mmHg for the threshold value for the mean arterial pressure TR_{MAP}, ±5 mmHg for the threshold value for the left ventricular end diastolic pressure TR_{LVEDP}, ±20 bpm for the threshold value for the heart rate TR_{HR} and ±75 msec for the threshold value for the heart rate variance TR_{HRV}. The controller 16 may be configured to use said threshold values TR_{CSRP} of the circulatory system related parameters CSRP as upper threshold values TRᵤₚₚₑᵣ and to calculate lower threshold values TR_{lower} therefrom e.g., by halving or doubling the threshold values TR_{CSRP} or by taking into account the age and the gender of the patient. Of course, it is also possible that the respective lower threshold values TR_{lower} are individually set by the physician. In the following exemplary embodiment, the lower threshold values TR_{lower} are set to be half or double of the respective upper threshold values TRᵤₚₚₑᵣ or TR_{CSRP} respectively.

Thereafter, the physician starts the weaning procedure in step S12. In particular, a signal is sent to the controller 16 to execute the weaning procedure. The controller 16 sets a first counter Cᵤₚ to zero and sets a second counter C_{down} to zero when the weaning procedure is executed initially. Further, the controller 16 defines a speed reduction value dN_{R} to correspond to the second speed reduction value dN_{R2} i.e., to 200 rpm.

In step S14, a first sequence of the heart rate HR, the heart rate variance HRV, the mean arterial pressure MAP and the left ventricular end diastolic pressure LVEDP is stored in the storage device 18 by the controller 16 for a first time interval. In this exemplary embodiment, the first sequence comprises a value for every one of the processed circulatory system related parameters CSRP for a timepoint every 2 sec within the first time interval of 30 min, for example. Hence, 900 values are stored for every one of the processed circulatory system related parameters CSRP in the storage device 18. The controller 16 may be configured to execute an outlier identification in the first sequence of the processed circulatory system related parameters CSRP.

Thereafter, the controller calculates a first average value AV₁ for the first sequence in step S16. In the embodiment shown in FIG. 2, a first average value for the first sequence of the heart rate HR, a first average value for the first sequence of the heart rate variance HRV, a first average value for the first sequence of the mean arterial pressure MAP and a first average value for the first sequence of the left ventricular diastolic pressure LVDEP is thus calculated.

After calculation of the first average values AV₁ the current set speed Nₛₑₜ is reduced by the speed reduction value dN_{R} in step S18. In the exemplary embodiment, the current set speed is 25,000 rpm which is thus reduced by 200 rpm. Hence, the new set speed Nₛₑₜ is 24,800 rpm.

After the reduction of the set speed Nₛₑₜ, a second sequence of the heart rate HR, the heart rate variance HRV, the mean arterial pressure MAP and the left ventricular end diastolic pressure LVEDP is stored in the storage device 18 by the controller 16 for a second time interval in step S20. In this exemplary embodiment, the second sequence comprises a value for every one of the processed circulatory system related parameters CSRP for a timepoint every 2 sec within the second time interval of 10 sec, for example. Hence, 5 values are stored for every one of the processed circulatory system related parameters CSRP in the storage device 18. The controller 16 may be configured to execute an outlier identification in the second sequence of the processed circulatory system related parameters CSRP.

Thereafter, the controller calculates a second average value AV₂ for the second sequence in step S22. In the embodiment shown in FIG. 2, a second average value for the second sequence of the heart rate HR, a second average value for the second sequence of the heart rate variance HRV, a second average value for the second sequence of the mean arterial pressure MAP and a second average value for the second sequence of the left ventricular diastolic pressure LVDEP is thus calculated.

In step S24, the controller 16 calculates comparison value |Diff| of the difference of the first average value AV₁ and the second average value AV₂ for every one of the circulatory system related parameters CSRP. In this exemplary embodiment, the comparison values for every one of the circulatory system related parameters CSRP are absolute values |Diff| for every one of the circulatory system related parameters CSRP. For instance, the first average value AV₁ for the heart rate HR may be 80.25 bpm and the second average value AV₂ for the heart rate HR may be 86.00 bpm so that the comparison value |Diff| for the heart rate is |80.25 bpm−86.00 bpm|=5.75 bpm.

The controller then compares the absolute value |Diff| with the respective threshold value TR_{CSRP} in step S26, in particular if the absolute value |Diff| lies within the threshold value TR_{CSRP}:
   |Diff| for heart rate HR≤TR_{HR}=20 bpm;
   |Diff| for heart rate variance HRV≥TR_{HRV}=75 msec;
   |Diff| for mean arterial pressure MAP≤TR_{MAP}=10 mmHg; and
   |Diff| for left ventricular end diastolic pressure LVEDP≤TR_{LVEDP}=5 mmHg.

Instead of comparing an absolute value |Diff| for every one of the circulatory system related parameters CSRP with the respective threshold value TR_{CSRP}, the comparison value may be calculated just as a difference. In this case, the respective threshold value TR_{CSRP} is a threshold range defined by the physician. For instance, the threshold value for the heart rate TR_{HR} may be defined as a threshold range of ±20 bpm. Staying with the above example for the heart rate HR, the comparison value is calculated as −5.75 bpm, which is within the threshold value TR_{HR} for the heart rate.

In step S26 it is established if the criteria are met and all absolute values |Diff| are within the threshold value TR_{CRSP} or not. If so, the first counter Cᵤₚ is increased by 1 by the controller 16 in step S28. In this regard it has to be emphasized that depending on the configuration of the controller 16 an absolute value |Diff| being equal to the respective threshold value TR_{CSRP} may be considered to be within the threshold or outside of the threshold.

Next, it is evaluated if the value of the first counter Cᵤₚ is below a predefined first counter maximum value TR_{Cup} in step S30. For instance, the predefined first counter maximum value TR_{Cup} may be 180. If the first counter Cᵤₚ is below the first counter maximum value TR_{Cup} the controller is configured to go back to step S20. If the first counter Cᵤₚ corresponds to the first counter maximum value TR_{Cup}, the controller is configured to evaluate if the set speed Nₛₑₜ corresponds to the minimum speed Nₘᵢₙ in step S32. If the set speed Nₛₑₜ corresponds to the minimum speed Nₘᵢₙ it is assumed that the patient is fully weaned and the blood pump 12 may be explanted. The weaning procedure is thus ended.

Optionally, the controller 16 may be configured to alter the set speed Nₛₑₜ to avoid regurgitation through the blood pump 12 prior to explantation of the blood pump 12. Thus, the blood pump 12 is then operated in a “zero flow” mode which delivers a further indication for the physician about the stability of the patient. An according process is described for instance in WO 2019/034775 A1 which is incorporated by reference.

In case the set speed Nₛₑₜ is above the minimum speed Nₘᵢₙ in step S32, the controller 16 sets the first counter Cᵤₚ to zero in step S34. The controller 16 than evaluates in step S36 if the absolute values |Diff| for all four circulatory system related parameters CSRP are within the lower threshold T_{lower} of the respective circulatory system related parameter CSRP. In particular, it is established whether the absolute value |Diff| for the heart rate HR is less or equal to 10 bpm, whether the absolute value |Diff| for the heart rate variance HRV is above or equal to 150 msec, whether the absolute value |Diff| for the mean arterial pressure MAP is less than or equal to 5 mmHg and whether the absolute value |Diff| for left ventricular end diastolic pressure LVEDP is less than or equal to 2.5 mmHg. If so, the controller 16 sets the speed reduction value dN_{R} to the first speed reduction value dN_{R1} in step S38 i.e., to 500 rpm. If not, the controller 16 sets the speed reduction value dN_{R} to the second speed reduction value dN_{R2} in step S40 i.e., to 200 rpm. After step S38 or S40 respectively the controller 16 returns to step S14.

If the controller 16 determines in step S26 that the absolute value |Diff| of at least one of the four circulatory system related parameters CSRP is not falling within the respective threshold value TR_{CSRP} the controller increases the second counter C_{down} by 1 in step S42.

Next, it is evaluated if the value of the second counter C_{down} is below a predefined second counter maximum value TR_{Cdown} in step S44. For instance, the predefined second counter maximum value TR_{Cdown} may be 12. In any case, the second counter maximum value TR_{Cdown} is distinctly less than the first counter maximum value TR_{Cup} by a factor of 10 to 20.

In case the value of the second counter C_{down} is less than the value of the second counter maximum value TR_{Cdown}, the controller 16 is configured to go back to step S20. If the value of the second counter C_{down} corresponds to the second counter maximum value TR_{Cdown}, the controller is configured to determine whether the current set speed Nₛₑₜ corresponds to the maximum speed Nₘₐₓ in step S46. If the set speed Nₛₑₜ corresponds to the maximum speed Nₘₐₓ it is assumed that the patient is not ready to be weaned and still needs full support of the blood pump 12. The weaning procedure is thus ended.

If the set speed Nₛₑₜ is below the maximum speed Nₘₐₓ, the controller 16 sets the second counter C_{down} to zero in step S48. Thereafter, the controller 16 determines if the speed increase value dN_{I} is to be altered in step S50. In particular, the controller 16 evaluates if one of the absolute values |Diff| calculated for the circulatory system related parameters CSRP is giving indication that the speed increase value dN_{I} is to be increased. Said indication may for instance be based on at least one of the distance between the absolute value |Diff| and the respective threshold value TR_{CSRP}, the value of the second counter Cᵤₚ, the frequency of the increase of the second counter Cᵤₚ or the second average value AV₂. If the speed increase value dN_{I} is to be increased, the controller 16 sets the speed increase value dN_{I} to the second speed increase value dN_{I2} in step S52.

If there is no indication for an increased speed increase value dN_{I}, the speed increase value dN_{I} is set to the first speed increase value dN_{I1} in step S54. After step S52 or S54 respectively, the controller 16 increases the set speed Nₛₑₜ by the speed increase value dN_{I} in step S56 and returns to step S20. The weaning procedure as described above in relation to FIG. 2 can further be amended without departing from the scope of the present invention. For instance, it is also possible to skip steps S36 to S40 and to apply only one predefined speed reduction value dN_{R}. Accordingly, it is also possible to skip steps S50 to S54 and to apply only one predefined speed reduction value dN_{R}. Further, the blood pump system 10 may be configured to suggest reasonable values for the parameters to be input by the physician in step S10, for instance based on the age and gender of the patient.

In addition, the controller 16 may be configured to evaluate in step S50 if a severe health condition of the patient is prevailing or expected e.g., based on the frequency of the increase of the second counter Cᵤₚ and the distances between all calculated absolute values |Diff| and the respective threshold values TR_{CSRP}. If such a severe condition is detected, the controller 16 may be configured to directly set the set speed Nₛₑₜ to the maximum speed Nₘₐₓ and to either end the weaning procedure or to go back to step S12. Of course, the controller 16 may also be configured to emit a signal to the physician in case such a severe condition is detected.

Alternatively to the above described exemplary embodiment, the physician may also define a minimum blood flow and a maximum blood flow instead of a minimum speed Nₘᵢₙ and a maximum speed Nₘₐₓ. Consequently, the first speed reduction value dN_{R1}, the second speed reduction value dN_{R2}, the first speed increase value dN_{I1} and the second speed increase value dN_{I2} then relate to a blood flow of the blood pump and not to a speed. Accordingly, the set speed Nₛₑₜ then corresponds to a set blood flow of the blood pump 12. In this regard, the at least one circulatory system related parameter CSRP may further comprise an average blood flow of the blood pump 12. In brief, the controller is then configured to reduce the speed stepwise from a current average blood flow of e.g., 2.5 l/min to a given average blood flow of e.g., 0.5 l/min (which corresponds to the defined minimum blood flow). When having reached the minimum average blood flow, the weaning procedure ends and the patient is ready for explantation of S the blood pump 12.

### Exemplary Implementations

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:
A1 Blood pump system comprising: a blood pump, in particular an intravascular blood pump, having a pump housing with a blood flow inlet and a blood flow outlet connected by a passage, a pump element disposed in said pump housing, a drive unit configured to drive the pump element at a set speed so that a flow is generated between the blood flow inlet and the blood flow outlet; a sensor arrangement configured to sense at least one circulatory system related parameter at least intermittently; a controller configured to process the at least one circulatory system related parameter; and a storage device configured to continuously store the processed at least one circulatory system related parameter; wherein the controller is further configured to execute a weaning procedure.
A2 Blood pump system according to paragraph A1, wherein the controller is further configured to execute the weaning procedure in that a first sequence of the processed at least one circulatory system related parameter is stored in the storage device for a first time interval.
A3 Blood pump system according to paragraph A2, wherein the controller is configured to calculate a first average value for the first sequence.
A4 Blood pump system according to paragraph A3, wherein the controller is further configured to reduce the set speed by a speed reduction value after the first average value is calculated.
A5 Blood pump system according to any one of the preceding paragraphs A1 to A4, wherein the controller is further configured to execute the weaning procedure in that a second sequence of the processed at least one circulatory system related parameter is stored in the storage device for a second time interval.
A6 Blood pump system according to paragraph A5, wherein the controller is configured to calculate a second average value for the second sequence.
A7 Blood pump system according to paragraph A6, wherein the controller is further configured to compare comparison value of the difference of the first average value and the second average value with a predefined threshold value.
A8 Blood pump system according to paragraph A7, wherein the controller is configured to increase a first counter by 1 provided the comparison value is within the predefined threshold value.
A9 Blood pump system according to paragraph A7 or A8, wherein the controller is configured to increase a second counter by 1 provided the comparison value is outside the predefined threshold value.
A10 Blood pump system according to any one of the preceding paragraphs A7 to A9, wherein the controller is configured to repeat the steps defined in paragraphs A5 to A9.
A11 Blood pump system according to any one of the preceding paragraphs A7 to A10, wherein the controller is configured to set the first counter to zero when the first counter reaches a predefined first counter maximum value.
A12 Blood pump system according to paragraph A11, wherein the controller is further configured to repeat the steps defined in paragraphs A2 to A11 when the first counter reaches a predefined first counter maximum value.
A13 Blood pump system according to any one of the preceding paragraphs A7 to A12, wherein the controller is configured to set the second counter to zero when the second counter reaches a predefined second counter maximum value.
A14 Blood pump system according to any one of the preceding paragraphs A7 to A13, wherein the controller is configured to increase the set speed by a speed increase value when the second counter reaches a predefined second counter maximum value.
A15 Blood pump system according to any one of the preceding paragraphs A7 to A14, wherein the controller is configured to repeat the steps defined in paragraphs A2 to A14 when the second counter reaches a predefined second counter maximum value.
A16 Blood pump system according to any one of the preceding paragraphs A1 to A15, wherein the controller is configured to end the weaning procedure when the set speed reaches a predefined minimum speed or a predefined maximum speed.
A17 Blood pump system according to any one of the preceding paragraphs A1 to A16, wherein the processed at least one circulatory system related parameter comprises one or more of a heart rate, a heart rate variance, a mean arterial pressure and a left ventricular end diastolic pressure.
A18 Blood pump system according to any one of the preceding paragraphs A7 to A17, wherein the predefined threshold value comprises one or more of a threshold value for the heart rate, a threshold value for the heart rate variance, a threshold value for the mean arterial pressure and a threshold value for the left ventricular end diastolic pressure.
A19 Blood pump system according to any one of the preceding paragraphs A11 to A19, wherein the first counter maximum value is larger than the second counter maximum value.
A20 Blood pump system according to paragraph A19, wherein the first counter maximum value is larger than the second counter maximum value by a factor of 10 to 20.
A21 Blood pump system according to any one of the preceding paragraphs A2 to A20, wherein the first time interval is larger than the second time interval.
A22 Blood pump system according to paragraph A21, wherein the first time interval is 30 min or more.
A23 Blood pump system according to paragraph A21 or A22, wherein the second time interval is 2 min or less.
A24 Blood pump system according to any one of the preceding paragraphs A7 to A23, wherein the predefined threshold value for the processed at least one circulatory system related parameter comprises a lower threshold range and an upper threshold range.
A25 Blood pump system according to paragraph A24, wherein the controller is configured to set the speed reduction value to a first speed reduction value when the comparison value is within the lower threshold range.
A26 Blood pump system according to paragraph A24 or A25, wherein the controller is configured to set the speed reduction value to a second speed reduction value when the comparison value is within the upper threshold range.
A27 Blood pump system according to paragraph A26, wherein the first speed reduction value is larger than the second speed reduction value.
A28 Blood pump system according to any one of the preceding paragraphs A4 to A27, wherein the speed reduction value is in a range between 100 rpm and 600 rpm.
A29 Blood pump system according to any one of the preceding paragraphs A26 to A28, wherein the first speed reduction value is in a range between 400 rpm to 600 rpm, wherein the first speed reduction value preferably is 500 rpm.
A30 Blood pump system according to any one of the preceding paragraphs A26 to A29, wherein the second speed reduction value is in a range between 100 rpm and 300 rpm, wherein the second speed reduction value preferably is 200 rpm.
A31 Blood pump system according to any one of the preceding paragraphs A11 to A30, wherein the controller is configured to set the speed increase value to a first speed increase value or to a second speed increase value based on the distance between the comparison value and the threshold value and/or based on the value of the second counter and/or based on the frequency of the increase of the second counter and/or based on the second average value.
A32 Blood pump system according to any one of the preceding paragraphs A11 to A31, wherein the speed increase value is in a range between 400 rpm and 1,500 rpm.
A33 Blood pump system according to paragraph A31 or A32, wherein the first speed increase value is in a range between 400 rpm to 600 rpm, wherein the first speed increase value preferably is 500 rpm.
A34 Blood pump system according to any one of the preceding paragraphs A31 to A33, wherein the second speed increase value is in a range between 800 rpm and 1,200 rpm, wherein the second speed increase value preferably is 1,000 rpm.
A35 Blood pump system according to any one of the preceding claims A2 to A34, wherein the first time interval is based on a number of heart beats.
A36 Blood pump system according to any one of the preceding claims A5 to A35, wherein the second time interval is based on a number of heart beats.
A37 Blood pump system according to any one of the preceding paragraphs A2 to A36, wherein the controller is further configured to execute an outlier identification in the first sequence of the processed at least one circulatory system related parameter.
A38 Blood pump system according to any one of the preceding paragraphs A2 to A37, wherein the controller is further configured to execute an outlier identification in the second sequence of the processed at least one circulatory system related parameter.
A39 Blood pump system according to any one of the preceding paragraphs A1 to A38, wherein the controller is configured to alter the set speed to avoid regurgitation through the blood pump.
A40 Blood pump system according to any one of the preceding paragraphs A1 to A39, wherein the comparison value is an absolute value.
B1 Method for automated weaning of a patient from a blood pump, in particular an intravascular blood pump, wherein the blood pump has a pump housing with a blood flow inlet and a blood flow outlet connected by a passage, a pump element disposed in said pump housing, a drive unit configured to drive the pump element at a set speed so that a flow is generated between the blood flow inlet and the blood flow outlet; wherein a sensor arrangement is configured to sense at least one circulatory system related parameter at least intermittently; wherein a controller is configured to process the at least one circulatory system related parameter; and wherein a storage device is configured to continuously store the processed at least one circulatory system related parameter; wherein controller is configured to execute the weaning method.
B2 Method according to paragraph B1, wherein a first sequence of the processed at least one circulatory system related parameter is stored in the storage device for a first time interval.
B3 Method according to paragraph B2, wherein a first average value for the first sequence is calculated.
B4 Method according to paragraph B3, wherein the set speed is reduced by a speed reduction value after the first average value is calculated.
B5 Method according to any one of the preceding paragraphs B1 to B4, wherein a second sequence of the processed at least one circulatory system related parameter is stored in the storage device for a second time interval.
B6 Method according to paragraph B5, wherein a second average value for the second sequence is calculated.
B7 Method according to paragraph B6, wherein an comparison value of the difference of the first average value and the second average value is compared with a predefined threshold value.
B8 Method according to paragraph B7, wherein first counter is increased by 1 provided the comparison value is within the predefined threshold value.
B9 Method according to paragraph B7 or B8, wherein a second counter is increased by 1 provided the comparison value is outside the predefined threshold value.
B10 Method according to any one of the preceding paragraphs B7 to B9, wherein the steps defined in paragraphs B5 to B9 are repeated.
B11 Method according to any one of the preceding paragraphs B7 to B10, wherein the first counter is set to zero when the first counter reaches a predefined first counter maximum value.
B12 Method according to paragraph B11, wherein the steps defined in paragraphs B2 to B11 are repeated when the first counter reaches a predefined first counter maximum value.
B13 Method according to any one of the preceding paragraphs B7 to B12, wherein the second counter is set to zero when the second counter reaches a predefined second counter maximum value.
B14 Method according to any one of the preceding paragraphs B7 to B13, wherein the set speed is increased by a speed increase value when the second counter reaches a predefined second counter maximum value.
B15 Method according to any one of the preceding paragraphs B7 to B14, wherein the steps defined in paragraphs B2 to B14 are repeated when the second counter reaches a predefined second counter maximum value.
B16 Method for automated weaning of a patient from a blood pump, in particular an intravascular blood pump, wherein the blood pump has a pump housing with a blood flow inlet and a blood flow outlet connected by a passage, a pump element disposed in said pump housing, a drive unit configured to drive the pump element at a set speed so that a flow is generated between the blood flow inlet and the blood flow outlet; wherein a sensor arrangement is configured to sense at least one circulatory system related parameter at least intermittently; wherein a controller is configured to process the at least one circulatory system related parameter; and wherein a storage device is configured to continuously store the processed at least one circulatory system related parameter; wherein the method comprises the following steps:
   i) a first sequence of the processed at least one circulatory system related parameter is stored in the storage device for a first time interval;
   ii) a first average value for the first sequence is calculated;
   iii) the set speed is reduced by a speed reduction value after the first average value is calculated;
   iv) a second sequence of the processed at least one circulatory system related parameter is stored in the storage device for a second time interval;
   v) a second average value for the second sequence is calculated;
   vi) a comparison value of the difference of the first average value and the second average value is compared with a predefined threshold value;
   vii) wherein the controller is configured to repeat steps iv) to vi), and configured to increase a first counter by 1 provided the absolute value is within the predefined threshold value, and configured to increase a second counter by 1 provided the comparison value is outside the predefined threshold value; and
wherein the controller is configured to repeat steps i) to vii) and configured to set the first counter to zero when the first counter reaches a predefined first counter maximum value, and wherein the controller is configured to increase the set speed by a speed increase value and configured to set the second counter to zero and configured to repeat steps iv) to vii) when the second counter reaches a predefined second counter maximum value.
B17 Method according to any one of the preceding paragraphs B1 to B16, wherein the weaning procedure is ended when the set speed reaches a predefined minimum speed or a predefined maximum speed.
B18 Method according to any one of the preceding paragraphs B1 to 17, wherein the processed at least one circulatory system related parameter comprises one or more of a heart rate, a heart rate variance, a mean arterial pressure and a left ventricular end diastolic pressure.
B19 Method according to any one of the preceding paragraphs B7 to B18, wherein the predefined threshold value comprises one or more of a threshold value for the heart rate, a threshold value for the heart rate variance, a threshold value for the mean arterial pressure and a threshold value for the left ventricular end diastolic pressure.
B20 Method according to any one of the preceding paragraphs B11 to B20, wherein the first counter maximum value is larger than the second counter maximum value.
B21 Method according to paragraph B20, wherein the first counter maximum value is larger than the second counter maximum value by a factor of 10 to 20.
B22 Method according to any one of the preceding paragraphs B2 to B21, wherein the first time interval is larger than the second time interval.
B23 Method according to paragraph B22, wherein the first time interval is 30 min or more.
B24 Method according to paragraph B22 or B23, wherein the second time interval is 2 min or less.
B25 Method according to any one of the preceding paragraphs B7 to B24, wherein the predefined threshold value for the processed at least one circulatory system related parameter comprises a lower threshold range and an upper threshold range.
B26 Method according to paragraph B25, wherein the speed reduction value is set to a first speed reduction value when the comparison value is within the lower threshold range.
B27 Method according to paragraph B25 or B26, wherein the speed reduction value is set to a second speed reduction value when the comparison value is within the upper threshold range.
B28 Method according to paragraph B27, wherein the first speed reduction value is larger than the second speed reduction value.
B29 Method according to any one of the preceding paragraphs B4 to B28, wherein the speed reduction value is in a range between 100 rpm and 600 rpm.
B30 Method according to any one of the preceding paragraphs B27 to B29, wherein the first speed reduction value is in a range between 400 rpm to 600 rpm, wherein the first speed reduction value preferably is 500 rpm.
B31 Method according to any one of the preceding paragraphs B27 to B30, wherein the second speed reduction value is in a range between 100 rpm and 300 rpm, wherein the second speed reduction value preferably is 200 rpm.
B32 Method according to any one of the preceding paragraphs B11 to B31, wherein the speed increase value is set to a first speed increase value or to a second speed increase value based on the distance between the comparison value and the threshold value and/or based on the value of the second counter and/or based on the frequency of the increase of the second counter and/or based on the second average value.
B33 Method according to any one of the preceding paragraphs B11 to B32, wherein the speed increase value is in a range between 400 rpm and 1,500 rpm.
B34 Method according to paragraph B32 or B33, wherein the first speed increase value is in a range between 400 rpm to 600 rpm, wherein the first speed increase value preferably is 500 rpm.
B35 Method according to any one of the preceding paragraphs B32 to B34, wherein the second speed increase value is in a range between 800 rpm and 1,200 rpm, wherein the second speed increase value preferably is 1,000 rpm.
B36 Method according to any one of the preceding claims B2 to B35, wherein the first time interval is based on a number of heart beats.
B37 Method according to any one of the preceding claims B5 to B36, wherein the second time interval is based on a number of heart beats.
B38 Method according to any one of the preceding paragraphs B2 to B37, wherein an outlier identification is executed in the first sequence of the processed at least one circulatory system related parameter.
B39 Method according to any one of the preceding paragraphs B2 to B38, wherein an outlier identification is executed in the second sequence of the processed at least one circulatory system related parameter.
B40 Method according to any one of the preceding paragraphs B1 to B39, wherein the set speed is altered to avoid regurgitation through the blood pump.
B41 Method according to any one of the preceding paragraphs B7 to B40, wherein the comparison value is an absolute value.

As utilized herein, the terms “approximately”, “about”, “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure. The terms “at least partially” or “partially” as used herein mean both partial and entirely or complete respectively.

1. A blood pump system comprising:
a blood pump, in particular an intravascular blood pump, having a pump housing with a blood flow inlet and a blood flow outlet connected by a passage, a pump element disposed in said pump housing, a drive unit configured to drive the pump element at a set speed (Nₛₑₜ) so that a flow is generated between the blood flow inlet and the blood flow outlet;
a sensor arrangement configured to sense at least one circulatory system related parameter (CSRP) at least intermittently;
a controller configured to process the at least one circulatory system related parameter (CSRP); and
a storage device configured to continuously store the processed at least one circulatory system related parameter (CSRP);
wherein the controller (16) is further configured to execute a weaning procedure in that
i) a first sequence of the processed at least one circulatory system related parameter (CSRP) is stored in the storage device (18) for a first time interval;
ii) a first average value (AV₁) for the first sequence is calculated;
iii) the set speed (Nₛₑₜ) is reduced by a speed reduction value (dN_{R}) after the first average value (AV₁) is calculated;
iv) a second sequence of the processed at least one circulatory system related parameter (CSRP) is stored in the storage device for a second time interval;
v) a second average value (AV₂) for the second sequence is calculated;
vi) a comparison value (|Diff|) of a difference of the first average value (AV₁) and the second average value (AV₂) is compared with a predefined threshold value (TR_{CSRP});
vii) wherein the controller is configured to repeat steps iv) to vi), and
configured to increase a first counter (Cᵤₚ) by 1 provided the comparison value (|Diff|) is within the predefined threshold value (TR_{CSRP}), and
configured to increase a second counter (C_{down}) by 1 provided the comparison value (|Diff|) is outside the predefined threshold value (TR_{CSRP}); and
wherein the controller (16) is configured to repeat steps i) to vii) and configured to set the first counter (Cᵤₚ) to zero when the first counter (Cᵤₚ) reaches a predefined first counter maximum value (TR_{Cup}), and
wherein the controller is configured to increase the set speed (Nₛₑₜ) by a speed increase value (dN₁) and configured to set the second counter (C_{down}) to zero and configured to repeat steps iv) to vii) when the second counter (C_{down}) reaches a predefined second counter maximum value (TR_{Cdown}).

2. A blood pump system according to claim 1, wherein the controller is configured to end the weaning procedure when the set speed (Nₛₑₜ) reaches a predefined minimum speed (Nₘᵢₙ) or a predefined maximum speed (Nₘₐₓ).

3. A blood pump system according to claim 1, wherein the processed at least one circulatory system related parameter (CSRP) comprises one or more of a heart rate (HR), a heart rate variance (HRV), a mean arterial pressure (MAP) and a left ventricular end diastolic pressure (LVEDP).

4. A blood pump system according to claim 3, wherein the predefined threshold value (TR_{CSRP}) comprises one or more of a threshold value for the heart rate (TR_{HR}), a threshold value for the heart rate variance (TR_{HRV}), a threshold value for the mean arterial pressure (TR_{MAP}) and a threshold value for the left ventricular end diastolic pressure (TR_{LVEDP}).

5. A blood pump system according to claim 1, wherein
the first counter maximum value (TR_{Cup}) is larger than the second counter maximum value (TR_{Cdown}).

6. A blood pump system according to claim 1, wherein
the first time interval is larger than the second time interval.

7. A blood pump system according to claim 4,
wherein the predefined threshold value (TR_{CSRP}) for the processed at least one circulatory system related parameter (CSRP) comprises a lower threshold range (TR_{lower}) and an upper threshold range (TRᵤₚₚₑᵣ).

8. A blood pump system according to claim 7, wherein the controller is configured to set the speed reduction value (dN_{R}) to a first speed reduction value (dN_{1R}) when the comparison value (|Diff|) is within the lower threshold range (TR_{lower}), and
configured to set the speed reduction value (dN_{R}) to a second speed reduction value (dN_{2R}) when the comparison value (|Diff|) is within the upper threshold range (TRᵤₚₚₑᵣ).

9. A blood pump system according to claim 8, wherein the first speed reduction value (dN_{1R}) is larger than the second speed reduction value (dN_{2R}).

10. A blood pump system according to claim 8, wherein
the speed reduction value (dN_{R}) is in a range between 100 rpm and 600 rpm, wherein the first speed reduction value (dN_{1R}) preferably is in a range between 400 rpm to 600 rpm and wherein the second speed reduction value (dN_{2R}) preferably is in a range between 100 rpm and 300 rpm.

11. A blood pump system according to claim 4, wherein
the controller is configured to set the speed increase value (dN₁) to a first speed increase value (dN₁₁) or to a second speed increase value (dN₂₁) based on a distance between the comparison value (|Diff|) and the predefined threshold value (TR_{CSRP}) and/or based on the value of the second counter (Cᵤₚ) and/or based on the second average value (AV₂).

12. A blood pump system according to claim 11, wherein
the speed increase value (dN_{I}) is in a range between 400 rpm and 1,500 rpm, wherein the first speed increase value (dN_{I1}) preferably is in a range between 400 rpm to 600 rpm and wherein the second speed increase value (dN_{I2}) preferably is in a range between 800 rpm and 1,200 rpm.

13. A blood pump system according to claim 1, wherein
the first time interval and the second time interval are based on a number of heart beats.

14. A blood pump system according to claim 1, wherein
the controller is further configured to execute an outlier identification in the first sequence of the processed at least one circulatory system related parameter (CSRP) and/or in the second sequence of the processed at least one circulatory system related parameter.

15. A blood pump system according to claim 1, wherein
the controller is configured to alter the set speed (Nₛₑₜ) to avoid regurgitation through the blood pump.
